(19) United States
(12) Patent Application Publication
(10) Pub. No.: US 2004/0082839 A1
(43) Pub. Date: Apr. 29, 2004

(54) System and method for mood contextual data output

(75) Inventor: Kenneth J. Haugen, Dakota Dunes, SD (US)

Correspondence Address: GATEWAY, INC., ATTN: SCOTT CHARLES RICHARDSON, 610 GATEWAY DR., Y-04, N. SIOUX CITY, SD, 57049, US

(73) Assignee: Gateway Inc.

(21) Appl. No.: 10/280,343

(22) Filed: Oct. 25, 2002

(51) Int. Cl. A61B 5/00

(52) U.S. Cl. 600/300

(57) ABSTRACT

The present invention is directed to a system and method for mood contextual data output. A method for providing mood contextual output from a user of an information handling system may include inferring a mood of a user based on biometric data collected while the user enters data on an information handling system. An output of data is affected based on the monitored user's mood.

### FIELD OF THE INVENTION

The present invention generally relates to the field of data input and output, and particularly to a system and method for mood contextual data output.

### BACKGROUND OF THE INVENTION

Email and other textual data have become more prevalent and relied upon by users of a wide range of information handling systems. From mobile phones and pagers to desktop and laptop computers, users are able to communicate over large distances in efficient and unobtrusive manner.

However, one of the problems in textual communication, and especially generated textual communication (such as typing), is that the recipient does not have a context for now the sender is communicating the text. A user may try to communicate a phrase that may be misinterpreted, by the person reading the communication because the reader does not know the mood of the user writing the text. Misunderstandings encountered in such instances may have disastrous results.

One of the methods currently utilized involves the arrangement of text by a user, such as alphanumeric and punctuation, to appear as faces having varying expressions, such as winking, smiling and the like “emotions”. This method is' informal and requires the user to enter the data, which may not be suitable in a wide variety of circumstances.

Therefore, it would be desirable to provide a system and method for mood contextual data output.

### SUMMARY OF THE INVENTION

Accordingly, the present invention is directed to a system and method for mood contextual data output. In an aspect of the present invention, a method for providing mood contextual output from a user of an information handling system includes monitoring a mood of a user while the user enters data on an information handling system. Data output is affected based on the monitored user's mood, the display or other output of data corresponding to the data entered by the user on the information handling system.

In an additional aspect of the present invention, a system for providing mood contextual output of data entered by a user includes a memory, an input device, an output device and a processor. The memory is suitable for storing a program of instructions. The input device is suitable for receiving data entered by a user or representing biometric data collected from a user and the output device is suitable for outputting data. The processor is communicatively coupled to the memory, the input device and the output device. The program of instructions configures the processor to monitor a mood of a user while the user operates the input device and affect a display or other output on data as displayed by the output device based on the monitored user's mood. The display of data may include the data entered by the user utilizing the input device.

In a further aspect of the present invention, a system for providing mood contextual output includes means for monitoring a mood of a user while the user enters data and means for affecting a display of data based on the monitored user's mood as received from the monitoring means. The display of data corresponds to the data entered by the user.

It is to be understood that both the forgoing general description and the following detailed description are exemplary and explanatory only and are not restrictive of the invention as claimed. The accompanying drawings, which are incorporated in and constitute a part of the specification, illustrate an embodiment of the invention and together with the general description, serve to explain the principles of the invention.

### BRIEF DESCRIPTION OF THE DRAWINGS

The numerous advantages of the present invention may be better understood by those skilled in the art by reference to the accompanying figures in which:

FIG. 1 is an illustration of an exemplary embodiment of the present invention wherein a system suitable for employing the present invention is shown;

FIG. 2 is a flow chart depicting an exemplary method of the present invention wherein a user's mood is monitored while entering data to affect a display of data entered by the user;

FIG. 3 is an illustration of an exemplary embodiment of the present invention wherein a display of data affected by a user's mood during entry of the data is shown;

FIG. 4 is an illustration of an exemplary embodiment of the present invention wherein a user's face as utilized for indicia of a user's mood is shown;

FIG. 5 is a flow chart of an exemplary method of the present invention wherein a user's body is monitored to detect a user's mood in order to affect a display of data;

FIG. 6 is a flow chart of an exemplary method of the present invention wherein a user's mood is monitored based on how data is entered by the user;

FIG. 7 is a flow chart depicting an exemplary method of the present invention wherein a reference for a user's mood is generated for comparison to a current user's mood;

FIG. 8 is an illustration of an exemplary embodiment of the present invention wherein data entered by a user is shown, the data suitable for indicating a mood of a user; and

FIG. 9 is a flow chart illustrating an exemplary method of the present invention wherein a user's mood is monitored based on data entered by the user.

### DETAILED DESCRIPTION OF THE INVENTION

Reference will now be made in detail to the presently preferred embodiments of the invention, examples of which are illustrated in the accompanying drawings.

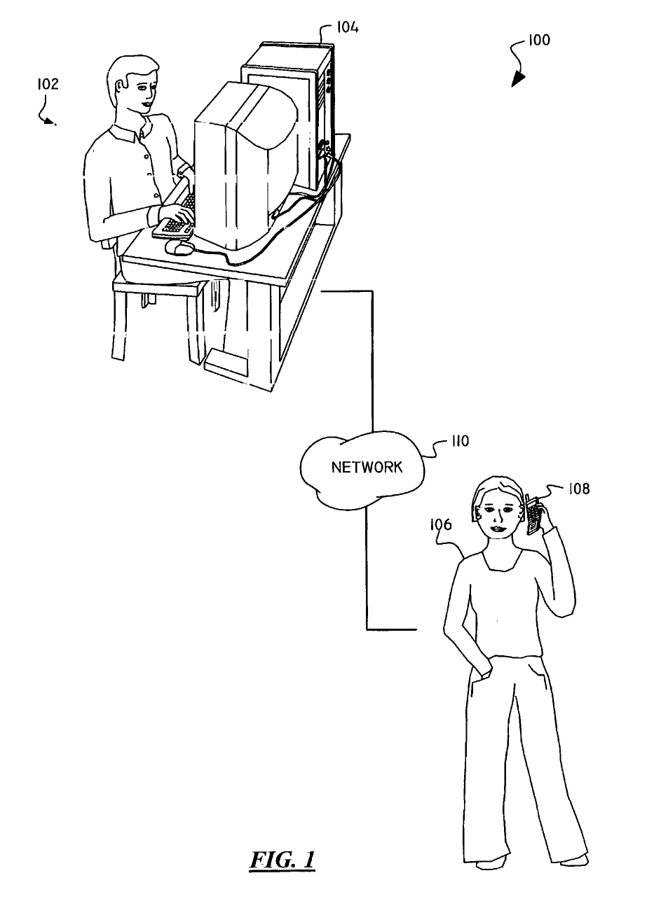

Referring generally now to FIGS. 1 through 9, exemplary embodiments of the present invention are shown. One of the problems in textual communication, and especially generated textual communications (such as typing), is that the user does not have a context for how the user is communicating the text. A user may try to communication a phrase that may be misinterpreted by the person reading the communication because the reader does not know the mood of the user writing the text. However, through use of the present invention, data input by a user may be placed in a mood context so that a person interacting with the data will have an indication of the user's mood when entering the data.

Referring now to FIG. 1, an exemplary embodiment 100 of the present invention is shown wherein a system for text messaging in accordance with the present invention is shown. A first user 102 may utilize an information handling system configured as a desktop computer 104 to communication with a second user 106 utilizing an information handling system configured as a wireless phone 108 over a network 110. Such communication, as previously described, has become more and more pervasive and enables people to communicate over great distances in a near instantaneous manner. A system embodying the present invention may provide instantaneous or time-delayed communication between the first and second users.

However, textual communication may lack “tone”, and therefore may not convey the mood of the user communicating, such as a statement made in jest, and the like. Therefore, communications between the first user 102 and the second user 106 may be subject to misinterpretation. Through use of the present invention, the user's mood is able to be monitored and conveyed to the person receiving the communication so that a greatly expanded method of communication is provided. In this way, users may communicate utilizing these pervasive forms of communication, such as text messaging, email, and the like, in an expanded, accurate and efficient manner.

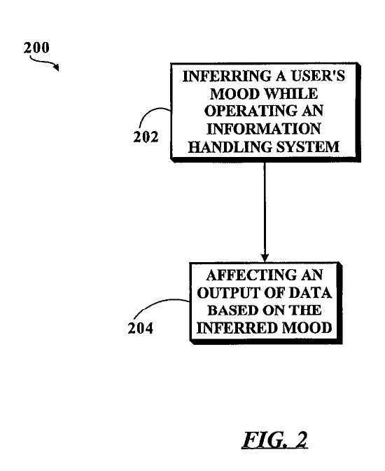

Referring now to FIG. 2, an exemplary method of the present invention is shown wherein a user's mood is monitored to affect a display of data which includes data entered by the user. A user's mood is monitored while the user enters data on an information handling system 202. A user may enter data utilizing a variety of methods, such as typing, handwriting recognition, speech, and the like as contemplated by a person of ordinary skill in the art.

A display of data corresponding to data as entered by the user is affected based on the inferred user's mood 204. Thus, the user's mood, i.e. the way the user feels at the time the data is entered, may provide context for the message entered by the user. A display of data may be affected based on mood utilizing a variety of methods without departing from the spirit and scope of the present invention. For example, a display may include audio clues or visual context clues. Visual context clues may include modifications to text font shape or size, changes to foreground or background color, or insertions of symbolic based clues.

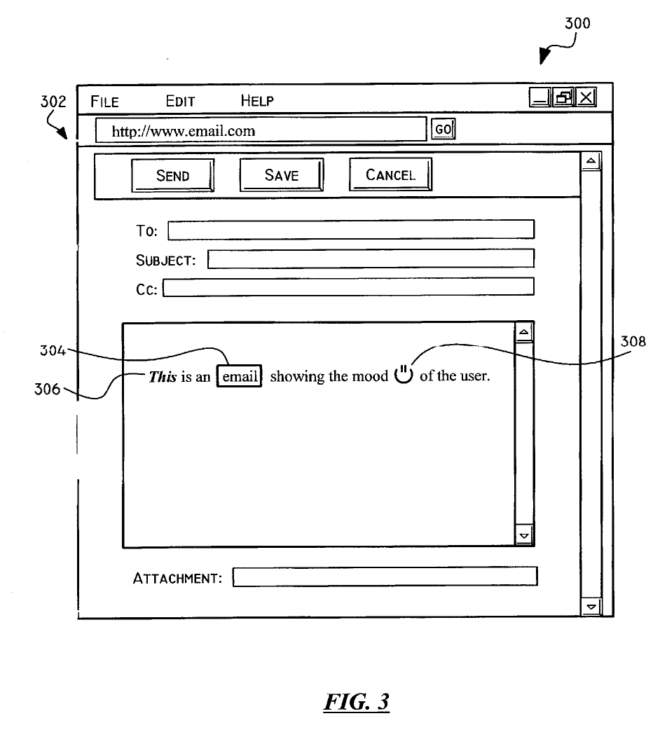

For example, referring now to FIG. 3, an exemplary embodiment 300 is shown in which a display of data is affected by a user's mood when entering the data. A user may enter data for a variety of purposes, such as an email 302. Data entered by the user may be affected to indicate the mood of the user, such as changing a portion of the display adjacent to the entered data 304. For example, the background of a display of text may be shown having different colors, textures, and the like to indicate a user's mood.

The data which was entered by the user may also be displayed in a manner to indicate the user's mood. For instance, the data may be displayed in varying fonts, treatments 306 such as bolding, underling, and italics, sizes of the data and fonts, and the like as contemplated by a person of ordinary skill in the art. Further, indicia may also be displayed to indicate mood, such as an icon, representation of a mood, such as a smiling face 308, and the like. In this way, a viewer of the data entered by the user may readily determine the user's mood to more efficiently and effectively understand the user's intentions.

Although text and the affecting a display of data as related to text has been described, a data output, as well as data entered by a user, may also include audio data, tactile data, and the like as contemplated by a person of ordinary skill in the art. For instance, a display of data may include audio data changed by tone, cadence, volume, and the like. A display of data may also include tactile data, such as affecting the feel of a particular portion of an information handling system, output of impulses to simulate a different “feel”, and the like without departing from the spirit and scope of the present invention. Finally, sounds may be played to indicate a happy, somber, angry or other like mood.

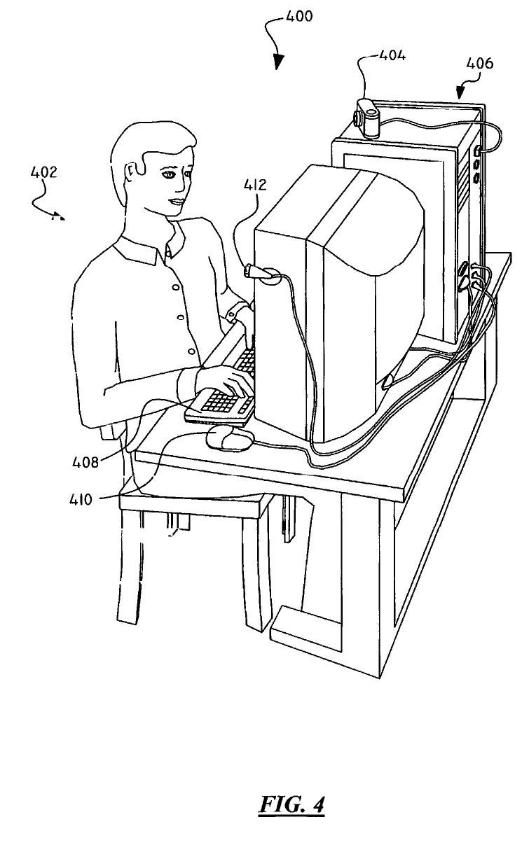

Referring now to FIG. 4, an exemplary embodiment 400 of the present invention is shown wherein a display of data is affected based on a user's mood is shown. A user 402 may express mood in a variety of ways based on the user's body. For instance, a user's skin temperature, differentiations in skin temperature (such as different temperatures of the face), body orientation (such as slouching, upright, eye dilation, twitching, and the like), body rhythms (such as heartbeat, breathing, brain waves, and the like) may be measured and analyzed to infer whether the user is agitated, angered, happy, relaxed, tensed, fearful and the like.

A variety of other mechanisms may also be employed to evaluate a user's mood, such as a camera 404 communicatively coupled to an information handling system 406 which is able to evaluate changes in a user's eye dilation, where the user is blushing, moving, or the like. Force sensors may be included in an input device 408 to evaluate the forcefulness of data entry by the user 402. Temperature and conductivity sensors may be included in a cursor control device 410 for further measurement of the user's mood. Additionally, a microphone 412 may be provided so as to enable the information handling system 406 to evaluate the user's voice during data entry. It should be realized that this listing is not meant to be exhaustive, and a variety of methods and devices for evaluating a user's mood are contemplated by the present invention without departing from the spirit and scope thereof. The data collected is collectively “user biometric data”.

Combinations of the user biometric data may be analyzed to infer the mood of a user. For example, a database may be provided for deriving a user's mood based on a variety of mood indications. For example, the database may include a look-up table which derives the mood of the user based on the following inputs.

| Inferred | | | | |
|---|---|---|---|---|
| Mood | Skin Temp. | Heart rate | Conductivity | Voice Level |
| Anger | Elevated | Elevated | Unchanged | Stressed |
| Tension | Unchanged | Unchanged or | Disregard | Elevated |
| | | Elevated | | |
| Fear | Unchanged | Elevated | Elevated | Unchanged or |
| | | | | Elevated |
| Happy/ | Disregard | Reduced | Disregard | Low |
| Relaxed | | | | |
| Happy/ | Disregard | Increased | Disregard | Low |
| Excited | | | | |
| Intoxi- | Increased | Disregard | Disregard | Low |
| cation | | | | |

Thus, a system employing the present invention uses a variety of inputs to determine the user's mood. In additional embodiments, the inputs may be weighted to give greater importance to more analytic or reliable indicia, a baseline emotion line created to compare values, and the like.

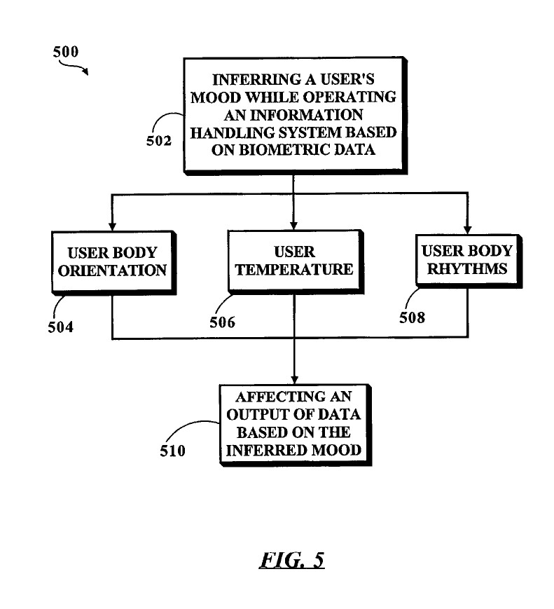

Referring now to FIG. 5, an exemplary method 500 of the present invention is shown wherein a user's mood is monitored based on the user's biometric data. A user's mood is inferred based on the user's biometric data while entering data on an information handling system, such as described in relation to FIG. 4. The mood as based on the user's body orientation may be indicated by the user's body orientation 504, user temperature 506, user body rhythms 508, and the like.

A display of data including data entered by the user is affected based on the monitored user's mood 510. In this way, passive information obtained from the user's body, i.e. information not directly entered by the user but rather obtained from the information handling system, may be utilized to improve communication. Thus, an efficient communication system is provided because the user is not required to enter additional information regarding mood of the user or require further explanation by the user when sending a communication.

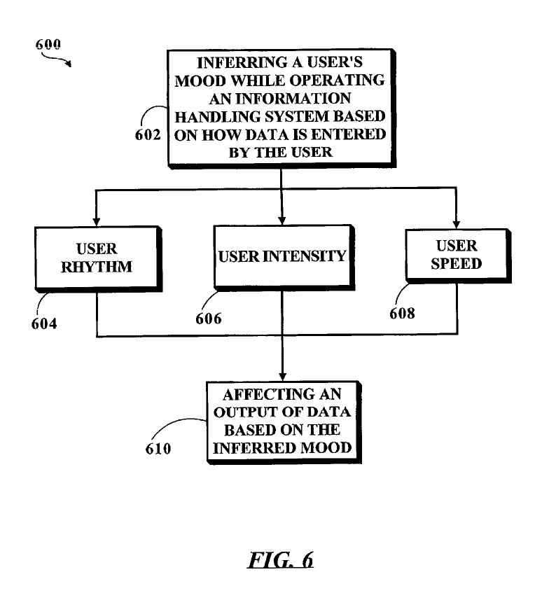

Referring now to FIG. 6, an exemplary embodiment 600 of the present invention is shown wherein a user's mood is inferred based on how data is entered by the user. A user's mood is inferred from user biometric data collected while the user enters data on an information handling system, based on how data is entered by the user 602. For example, the user's rhythm of data entry 604 may be indicative of the user's mood, such when the user speeds up when typing a first second of an email as opposed to a second section of an email.

The user's intensity 606 may also indicate the user's mood, such as how hard the user presses keys when typing, force of a pen stroke on a touch screen, and the like. Further, a user's speed 608 of entering data may also indicate the user's mood, such as agitation or nervousness on the part of the user. A display of data as entered by the user may then be affected based on the monitored mood 610 as previously described.

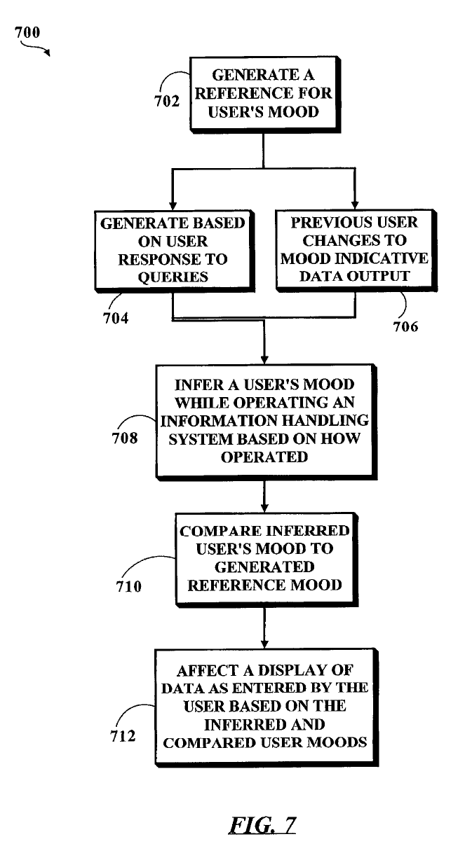

One method of a variety of methods utilized to determine the user's mood includes utilizing a baseline reference of a user's mood to determine mood changes and the overall mood of the user. For example, referring now to FIG. 7, an exemplary method 700 of the present invention is shown wherein a generated baseline is utilized to determinate a user's mood. A reference for a user's mood is generated, 702 which is suitable for comparison with monitoring performed by an information handling system to determine the user's mood. A reference mood may be generated based on a user's response to queries, previous user changes to mood indicative display of data 706, and the like. Thus, once a user's mood is inferred while operating an information handling system as previously described 708, the inferred user's mood may be compared to the generated referenced mood 710. The output of data is affected as before 712.

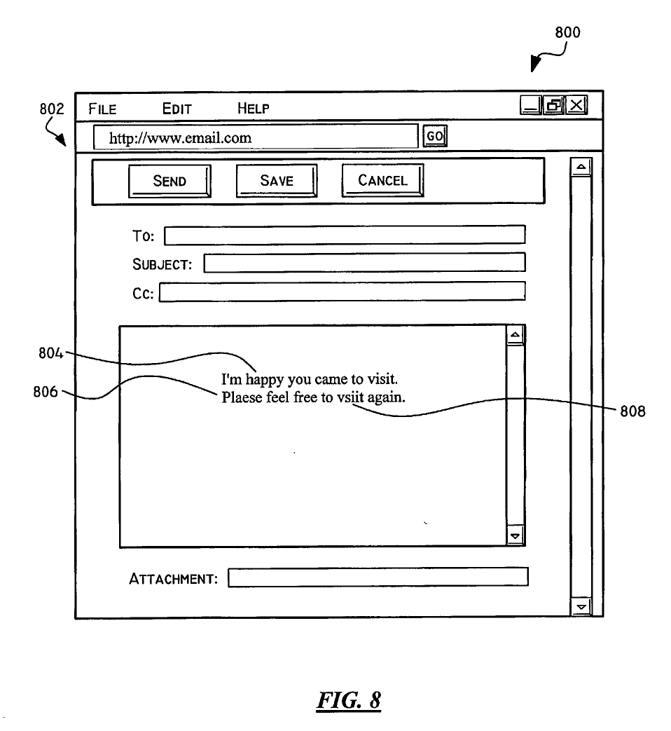

Referring now to FIG. 8, an exemplary embodiment 800 of the present invention is shown wherein a user's mood is monitored based on the data entered by the user. A user, when entering data, may indicate mood based on the data entered. For instance, an email 802 may contain words which indicate the user's mood directly, such as “angry”, “mad”, “happy” 804, and the like. Additionally, even misspelled 806 words, and the frequency of the misspelling 806 & 808, may indicate mood, such as agitation on the part of the user, and the like. Therefore, the words used, arrangement of the words, misspelled words, and the like, may be utilized to determine a user's mood.

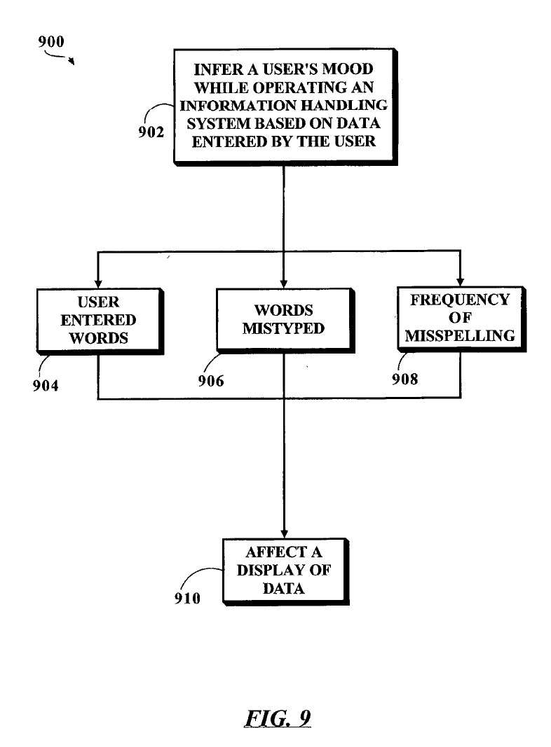

For example, as shown in the exemplary method 900 depicted in FIG. 9, a user's mood may be determined by the data entered by the user. A user's mood is monitored while the user enters data on an information handling system, based on the data entered by the user 902. The data entered may include the entered words 904, words mistyped by the user 906, frequency of misspelling 908, and the like. A display of data is then affected based on the monitored user's mood. In this way, the present invention may provide indicia beyond the actual words entered by the user to indicate the user's mod at the time the user entered the data.

In exemplary embodiments, the methods disclosed may be implemented as sets of instructions or software readable by a device. Further, it is understood that the specific order or hierarchy of steps in the methods disclosed are examples of exemplary approaches. Based upon design preferences, it is understood that that specific order or hierarchy of steps in the method can be rearranged while remaining within the scope of the present invention. The accompanying method claims present elements of the various steps in a sample order, and are not necessarily meant to be limited to the specific order or hierarchy presented.

It is believed that the system and method of the present invention and many of its attendant advantages will be understood by the forgoing description. It is also believed that it will be apparent that various changes may be made in the form, construction and arrangement of the components thereof without departing from the scope and spirit of the invention or without sacrificing all of its material advantages. The form herein before described being merely an explanatory embodiment thereof. It is the intention of the following claims to encompass and include such changes.

What is claimed is:

1. A method for providing mood contextual output from an information handling system, comprising the steps of:
inferring a mood of a user while the user operates an information handling system; and
affecting an output of data based on the inferred mood.

2. The method as described in claim 1, wherein the display of data corresponds to the data entered by the user on the information handling system.

3. The method as described in claim 1, wherein the inferred mood is an emotion the user feels at a particular time the user operates the information handling system.

4. The method as described in claim 1, wherein the mood of the user is inferred from the user's biometric data.

5. The method as described in claim 4, wherein the mood of the user is inferred from at least one of user body orientation, user body temperature, user body rhythm, user skim conductivity, user heart rate, user breathing, user brain waves and user voice stress.

6. The method as described in claim 1, wherein the mood of the user is inferred from how data is entered by the user on the information handling system.

7. The method as described in claim 6, wherein how data is entered includes at least one of rhythm of data entry, intensity of data entry and sped of data entry.

8. The method as described in claim 1, wherein the mood of the user is inferred from the data entered by the user on the information handling system.

9. The method as described in claim 7, wherein the mood is inferred from at least one of user entered words, words mistyped by the user and frequency of misspelling by the user.

10. The method as described in claim 1, further comprising the step of comparing an inferred mood to a generated reference mood.

11. The method as described in claim 1, wherein the user operates the information handling system through audio means.

12. The method as described in claim 1, wherein the affected display of data includes at least one of audio and tactile data.

13. A system for providing mood contextual output of data, comprising:
a memory for storing a program of instructions;
input device for receiving data entered by a user;
an output device for outputting a display of data; and
a processor communicatively coupled to the memory, the input device and the output device, wherein the program of instructions configures the processor to:
infer a mood of a user while the user uses the input device; and
affect a display of data by the output device based on the inferred mood, the display of data including the data entered by the user utilizing the input device.

14. The system as described in claim 13, wherein the inferred mood is an emotion the user feels at a particular time the user operates the information handling system.

15. The system as described in claim 13, wherein the mood of the user is inferred based on the user's biometric data.

16. The system as described in claim 15, wherein the mood of the user is inferred from at least one of user body orientation, user temperature, user body rhythm, user skin conductivity, user heart rate, user breathing, user brain waves and user voice stress.

17. The system as described in claim 13, wherein the mood of the user is inferred from how data is entered by the user on the information handling system.

18. The system as described in claim 17, wherein how data is entered includes at least one of rhythm of data entry, intensity of data entry and speed of data entry.

19. The system as described in claim 13, wherein the mood of the user is inferred based on the data entered by the user on the information handling system.

20. The system as described in claim 19, wherein the mood is monitored based on at least one of user entered words, words mistyped by the user and frequency of misspelling by the user.

21. The system as described in claim 13, wherein the program of instructions further configures the processor to compare an inferred user's mood to a generated reference mood.

22. The system as described in claim 13, wherein the input devices enables a user to operate the information handling system through audio means.

23. The system as described in claim 13, wherein the output device is suitable for outputting at least one of audio and tactile data.

24. A system for providing mood contextual output, comprising:
means for monitoring biometric data of a user while the user enters data; and
means for affecting a display of data based on the monitored user's mood as inferred from the monitored biometric data.

25. The system as described in claim 24, wherein the inferred mood is an emotion the user feels at a particular time the user entered the data.

26. The system as described in claim 25, wherein the mood of the user is inferred from at least one of user body orientation, user temperature, user body rhythm, user skin conductivity, user heart rate, user breathing, user brain waves and user voice stress.

27. The system as described in claim 24, wherein the monitoring means infers the mood of the user based on how data is entered by the user.

28. The system as described in claim 27, wherein how data is entered includes at least one of rhythm of data entry, intensity of data entry and speed of data entry.

29. The system as described in claim 24, wherein the monitoring means infers the mood of the user based on the data entered by the user.

30. The system as described in claim 29, wherein the mood is inferred from at least one of user entered words, words mistyped by the user and frequency of misspelling by the user.

31. The system as described in claim 24, further comprising a means for comparing a monitored user's mood to a generated reference mood.

32. The system as described in claim 24, wherein the system includes means for entering audio data so that the user enters data through the audio means to the information handling system.

33. The system as described in claim 24, wherein the affected display of data includes at least one of means for outputting a display of audio data and means for outputting a display of tactile data.
